(19) United States
(12) Patent Application Publication
(10) Pub. No.: US 2010/0076425 A1
(43) Pub. Date: Mar. 25, 2010

(54) UROLOGICAL BALLOON CATHETER

(76) Inventor: Alexander CARROUX, Pinneberg (DE)

Correspondence Address: OSTROLENK FABER GERB & SOFFEN, 1180 AVENUE OF THE AMERICAS, NEW YORK, NY, 100368403, US

(21) Appl. No.: 12/547,902

(22) Filed: Aug. 26, 2009

(30) Foreign Application Priority Data
Sep. 23, 2008 (DE) 102008048616.7

(51) Int. Cl. A61B 18/18

(52) U.S. Cl. 606/41

(57) ABSTRACT

A urological balloon catheter has electrodes in a region of its shaft proximally adjoining the balloon, which electrodes lie uncovered on the outer surface of the shaft and can be connected to a high-frequency generator via lines which run through the shaft in the proximal direction.

### BACKGROUND OF THE INVENTION

Balloon catheters, such as those used in many different applications in urology, have an internal discharge channel, running from the distal tip to the proximal end, and a balloon surrounding the shaft of the catheter, usually arranged at a small distance from the distal tip, which balloon can be inflated with a suitable liquid or gaseous medium through an inflation channel which runs separately through the catheter to the proximal end region.

Balloon catheters are passed through the urethra into the bladder. The balloon is then inflated in the bladder and secures the balloon catheter against slipping out.

The balloon catheter, positioned with the balloon inflated, lies securely in a fixed axial position in the urethra and its distal tip lies in the bladder. It ensures an undisturbed outflow of urine from the bladder and is used after operations or in the case of outflow disorders which are caused, for example, by a hypertrophic prostate.

The hypertrophic prostate can be treated very effectively and with little strain to the patient using the transurethral resection. In the process, a specific endoscope is used to bring a cutting instrument into the region of the prostate through the urethra in order to strip the prostate there from the inside. The cutting instruments are usually high-frequency-actuated cutting blades or else lasers. A disadvantage in these methods is however the risk of severe bleeding, which requires a relatively long stay in hospital. Furthermore, these methods are only bearable for the patient under anesthetics and so there is a by all means significant anesthetics risk for older patients.

This usual treatment of the prostate by transurethral resection is very effective but is also a great strain on the patients who usually have a very advanced age. It is for this reason that solutions, in the form of less stressful operations, which do not necessarily have to have the same long-term effect have been sought for a relatively long time.

By way of example, catheters passed through the urethra are known which destroy tissue in the region of the prostate by heating, e.g. by using microwaves. However, in this method, the volume reduction that can be attained in the case of the tissue to be ablated is too low.

German Publication DE 10 2007 010 076 A1 describes a shaft of a catheter with electrode rings on the outer surface. All the rings are connected to one pole of the high-frequency generator. The other pole of the high-frequency generator is connected to a separate electrode on a rectal catheter. Current therefore flows between the electrodes on the balloon catheter and the rectal catheter through the body tissue.

German Publication DE 10 2004 055 866 A1, in FIGS. 9-10d, shows a catheter with ring electrodes on the distal end region. The catheter has no balloon and therefore has to be placed carefully with the electrode rings inside the prostate. The electrode rings are connected to the poles of the high-frequency generator such that current always flows only between specific electrodes. The catheter has to be moved in the axial direction to apply current flow uniformly to the field of application.

### SUMMARY OF THE INVENTION

The object of the present invention is to permit a treatment of the prostate which is not very stressful.

This object is achieved by the features disclosed herein.

The balloon catheter according to the invention first of all has the positive characteristics of the balloon catheter: to be able to be inserted into the urethra without relatively large amounts of pain, i.e. without anesthetics, because of its flexible shaft. It is held at a defined location by means of the balloon and then sits with a particular part of its length, which proximally adjoins the balloon, in the region of the prostate. According to the invention, electrodes are arranged at that location on the balloon catheter, the electrodes being high-frequency-actuated and hence destroying tissue in their surroundings. Prostate tissue can thus be removed and a channel can be opened up around the catheter in the prostate which, after completion of the operation and removal of the balloon catheter, once again ensures the normal urine outflow. However, prostate tissue is removed only in a relatively tight region around the surface of the balloon catheter. Thus, the operation does not cause a lot of strain and, in particular, passes without relatively large amounts of bleeding. The treatment can be performed such that work is conducted practically without bleeding and the treated surfaces are cauterized, i.e. closed. The balloon catheter can be removed directly after the operation and the patient can be discharged. This results in optimum rest, which is very valuable, particularly in older patients.

According to the invention, current is flowing between alternate electrodes so that over the length of the part of the shaft bearing the electrodes, a more or less uniform current-flow is applied to the surrounding prostate tissue. No axial movement of the catheter is needed to make the effects of the current uniform.

The electrodes can be arranged in a more or less arbitrary fashion on the surface of the catheter, for example as dots which are arranged in the region of the prostate with an arbitrary pattern. However, the electrodes preferably have an annular design, which makes a clearly arranged current guidance possible. In the process, the electrodes are preferably connected alternately to the two poles so that a very even plasma density can be obtained above the region provided with the electrodes.

The electrodes can be connectable in a permanent fashion. However, they are preferably connected via switches, by means of which the electrodes can be connected or switched off individually or in groups. This affords the possibility of, for example, switching off certain electrodes in order to protect certain tissue regions. For example, if the electrodes are arranged over a relatively long length region of the shaft in order to be able to also treat a very large prostate, and this balloon catheter is now intended to be used in the case of a very short prostate, the electrodes which are not required can be switched off so as not to damage healthy tissue such as the sphincter muscle which distally adjoins the prostate.

The balloon catheter is usually composed of elastic material with a certain flexibility and is surrounded with pressure by the hypertrophic prostate after it has been inserted, as a result of which the prostate is pushed against the region with the electrodes until sufficient amounts of tissue have been removed from that location. This causes very little strain to the patient but often only causes temporary relief because the prostate can grow back. The operation can then be repeated from time to time; this should still be preferred to a radical, very stressful prostate treatment if the patient can only be subjected to small amounts of strain. The interval until the next treatment can be increased if the balloon catheter is designed wherein at least the region of the shaft which supports the electrodes is designed such that its circumference can be increased. At least that region of the balloon catheter in which the electrodes are provided can be designed such that its circumference can be increased and it can then be inflated during the operation in order to remove prostate tissue in a larger circumferential region. The circumference can be increased, for example, by inflating in a similar fashion as in the region of the balloon.

The tissue ablating work of the electrodes assumes a current flow between the electrodes. The current must flow through a liquid which must be able to conduct. If the catheter sits too tightly in the prostate, liquid is missing in this case between the surface of the catheter and the abutting tissue. A sufficient current flow cannot form in this case. As a result of the openings in the region of the electrodes, rinsing fluid can be inserted directly into the working region of the electrodes and so the correct current flow is ensured.

### BRIEF DESCRIPTION OF THE DRAWINGS

In the drawing, the invention is illustrated in an exemplary and schematic fashion. In the figures:

FIG. 1 shows a section through the region of a prostate with a balloon catheter according to the invention,

FIG. 2 shows a side view of the balloon catheter of FIG. 1,

FIG. 3 shows a section in accordance with the line 3-3 in FIG. 2,

FIG. 4 shows a side view of a region of the catheter according to the invention in a different embodiment,

FIG. 5 shows a portion of FIG. 2, indicating the electrical polarities,

FIG. 6a shows a side view similar to that of FIG. 4 in a different embodiment, and

FIG. 6b shows the side view of FIG. 6a in partially inflated state.

### DETAILED DESCRIPTION OF THE INVENTION

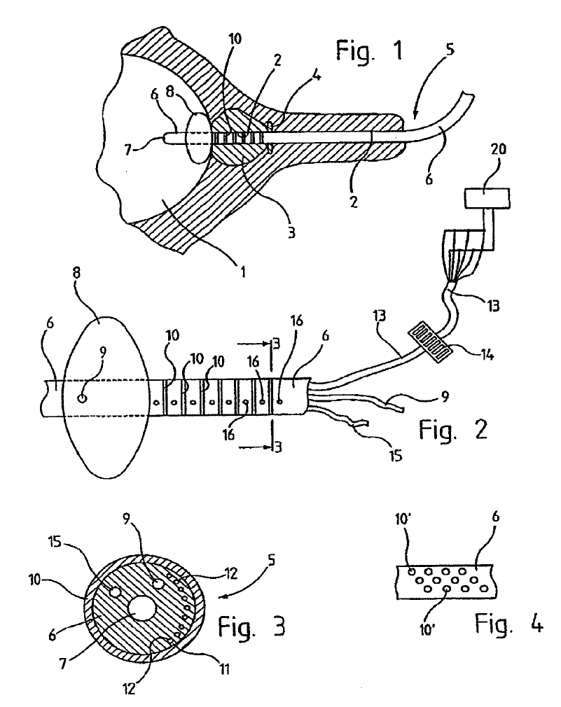

FIG. 1 shows a human bladder 1 which is connected to the outside by the urethra 2 which, along its way, passes through the prostate 3 and the outer sphincter muscle 4.

In the urethra 2 there is a balloon catheter 5 with an elongate, elastically flexible shaft 6 which is pervaded along its entire length by a discharge channel 7. The opening of the discharge channel 7 at the distal end of the shaft 6 can be seen in FIG. 1, while FIG. 3 shows its position in the catheter in a sectional view. The discharge channel 7 is used to empty or rinse the bladder 1, but it can also be absent in the use according to the invention.

At a small distance from the distal tip, the catheter 6 supports an encircling balloon 8 which is connected to an inflation channel 9, the latter being illustrated in FIG. 2 with its opening into the balloon and in FIG. 3 with its position in the cross section of the shaft 6. FIG. 2 shows that it runs in the proximal direction and is connected there, in a fashion which is not illustrated, to, for example, a piston syringe by means of which a pressure medium for inflation can be inserted into the balloon 8.

The emptied balloon lies flat against the catheter 6 (not illustrated). In this position, the balloon can be brought into the position illustrated in FIG. 1 through the urethra 2 without any problems. Thereupon, the balloon 8 is inflated into the shown position and then blocked by closing off the inflation channel 9 using a valve (not illustrated). The balloon catheter 5 now lies securely in the position illustrated in FIG. 1.

In this position, a length region of the balloon catheter 5, which adjoins the balloon 8 proximally, lies within the prostrate 3. The catheter is provided with electrodes 10 in this length region, which electrodes are formed annularly around the axis of the catheter 6 in the exemplary embodiment of FIGS. 1 to 3.

A number of such annular electrodes 10 are arranged one behind the other. One of these is shown in a sectional view in FIG. 3.

Every electrode is individually connected in the exemplary embodiment. The section in FIG. 3 shows that the electrode illustrated in a section there is connected to one of a number of parallel running lines 12 via a short feed line 11, the lines 12 running out of the proximal end of the catheter 6 as a bundle of lines 13 and to a current source in the form of the illustrated high-frequency generator 20. The other electrodes are connected just as illustrated in FIG. 3. Thus, each electrode ring 10 is connected to a dedicated line 12 which leads to the outside.

In the exemplary embodiment, the bundle of lines 13 runs via a switch 14 where, for example, each of the individual lines 12 can be switched by means of the illustrated individual switching elements. Using this, it is for example possible, in the configuration illustrated in FIG. 1, to switch off the proximally outermost electrode ring 10 which lies within the outer sphincter muscle 4, in order to avoid damage to the sphincter muscle, which could lead to incontinence.

The electrode rings 10 are preferably connected to the high-frequency generator (20) such that in the longitudinal direction of the catheter 10 they are always alternately connected to one or the other pole of the current source. Electrodes 10 lying one behind the other in the axial direction thus have differing polarity. It follows that a current flows between respectively adjacent electrodes.

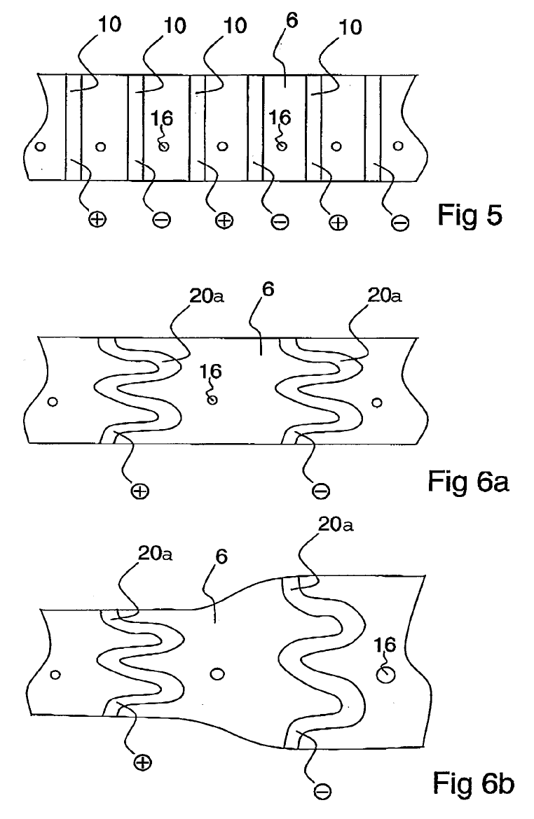

FIG. 5 shows a portion of FIG. 2 indicating the polarities +, −, +, −, +, − of the alternately connected electrode rings 10.

An additional inlet channel 15 is provided in the catheter 6, which inlet channel supplies a number of openings in the surface of the catheter 6 which are arranged in the region of the electrodes 10. If the rinsing fluid is inserted with a little pressure, it can be distributed between the catheter and the tissue, even in the case of tightly abutting tissue, and it can ensure a sufficient current flow.

An alternative possibility for arranging the electrodes is illustrated in FIG. 4. Here the electrodes 10′ are arranged in punctiform fashion, and not annularly, as is the case of electrodes 10 in the embodiment of FIGS. 1-3. Here, it also needs to be ensured that, in the case of a bipolar operation, the electrodes are connected alternately to the two poles.

A mono-polar connection of the electrodes is also possible. In that case, all electrodes are to be connected to one pole of the current source, while the other pole of the current source is connected to a plate electrode which contacts the surface of the patient in a conducting fashion.

In the case of the preferred bipolar operation, current flows between neighboring electrodes 10 and 10′ which have different polarity. A plasma is formed in the region of the electrodes, said plasma vaporizing the abutting tissue to a small penetration depth. The prostate undergoing hypotrophy and pressing with pressure against the catheter can relax as tissue is ablated internally. An open channel remains after the catheter is removed.

The diameter of this channel can be enlarged if the diameter of the shaft 6 of the catheter 5 increases when the electrodes are switched on. This can for example be achieved if the catheter is inflated at least in the region provided with the electrodes. For example, the distal end of the discharge channel 7 can be closed off to this end and the channel 7, or another suitable cavity, can be filled with a pressure medium under high pressure.

FIG. 6a shows a similar region of shaft 6 as represented in FIG. 5. The only difference is that the ring electrodes 20a of this embodiment are not wound in coaxial circles around the axis of shaft 6, but instead are arranged in meandering form. As shown in FIG. 6b, the meander-like form of the ring electrodes 20a more easily will bear the tension stress when the diameter of the shaft 6 is widened up.

FIG. 6b shows a partly inflated state of shaft 6. Part of the shaft remains at the smaller diameter of FIG. 6a whereas another part is widened up considerably. In this way, the outer diameter of the shaft may follow the uneven local conditions of the surrounding prostate.

Although specific embodiments have been illustrated and described herein for purposes of description of the preferred embodiment, it will be appreciated by those ordinary skilled in the art that a wide variety of alternate and/or equivalent implementations calculated to achieve the same purposes may be substituted for the specific embodiment shown and described without departing from the scope of the present invention. This application is intended to cover any adaptations or variations of the embodiments discussed herein. Therefore, it is manifestly intended that this invention only be limited by the appended claims and the equivalents thereof.

What is claimed is:

1. A urological balloon catheter which has electrodes in a region of its shaft proximally adjoining the balloon, which electrodes lie uncovered on the outer surface of the shaft and can be connected to a high-frequency generator via lines which run through the shaft in the proximal direction, wherein, in the axial direction, the electrodes can be connected alternately to one or the other of the two poles of the high-frequency generator.

2. The balloon catheter according to claim 1, wherein the electrodes are formed annularly around the axis of the shaft and are connected, one behind the other, alternately to one or the other of the two poles of the high-frequency generator.

3. The balloon catheter according to claim 1, wherein the electrodes can be connected individually or in groups by means of switches.

4. The balloon catheter according to claim 1, wherein at least the region of the shaft which supports the electrodes is designed such that its circumference can be increased.

5. The balloon catheter according to claim 1, wherein the region of the shaft which supports the electrodes is provided with openings connected to an inlet of conductive rinsing fluid.

6. A method of treating the human prostate with high-frequency current, comprising:
providing a urological balloon catheter with electrodes on the outer surface of the shaft;
connecting the electrodes alternately to one or the other of the two poles of the high-frequency generator;
placing the balloon catheter with its electrodes inside the prostate; and
keeping the balloon catheter in a fixed position while having current flow between the electrodes.

7. A method of treating the human prostate with high-frequency current, comprising:
providing a urological balloon catheter with electrodes on the outer surface of the shaft;
connecting the electrodes alternately to one or the other of the two poles of the high-frequency generator;
arranging openings on the region of the shaft supporting the electrodes;
connecting the openings to an inlet of conductive rinsing fluid; and
letting the fluid flow out of the openings while electrical current is flowing between the electrodes.

8. A method of treating human prostate with high-frequency current, comprising:
providing a urological balloon catheter with electrodes on the outer surface of the shaft;
connecting the electrodes alternately to one or the other of the two poles of the high-frequency generator;
inflating at least the region of the balloon catheter in which the electrodes are provided; and
inflating the region during the operation while electrical current is flowing between the electrodes.
